(19) United States
(12) Patent Application Publication
(10) Pub. No.: US 2025/0195472 A1
(43) Pub. Date: Jun. 19, 2025

(54) Indole Alkaloid as Antineoplastic Agent

(71) Applicant: NAPP TECNOLOGIAS, S.A. DE C.V., Durango (MX)

(72) Inventors: Jesús Alejandro Dominguez Puente, Durango (MX); José Manuel Narváez Mastache, Durango (MX)

(21) Appl. No.: 18/688,673

(22) PCT Filed: Aug. 30, 2022
(86) PCT No.: PCT/MX2022/050071

§ 371 Date: Mar. 3, 2025

(30) Foreign Application Priority Data
Sep. 1, 2021 (MX) MX/A/2021/010532

(51) Int. Cl. A61K 31/407; A61P 35/00

(52) U.S. Cl. CPC A61K 31/407; A61P 35/00

(57) ABSTRACT

An indole alkaloid, specifically the alkaloid (3S,3aR,8aS)-3-butyl-5-hydroxy-3,3a,8a-trimethyl-3,3a,8,8a-tetrahydro-2H-furo[2,3-b]indole-2-one, with the generic name Andranone, as an antineoplastic agent against neoplastic cells of colon cancer, breast cancer, leukaemia, central nervous system cancer, prostate cancer, lung cancer and cervical cancer.

### CROSS-REFERENCE TO RELATED APPLICATIONS

This application is the United States national phase of International Patent Application No. PCT/MX2022/050071 filed Aug. 30, 2022, and claims priority to Mexican Patent Application No. MX/a/2021/010532 filed Sep. 1, 2021, the disclosures of which are hereby incorporated by reference in their entireties.

### BACKGROUND

### Technical Field

The present disclosure relates to an indole alkaloid as an antineoplastic agent, and more particularly relates to the alkaloid (3S, 3aR, BaS)-3-butyl-5-hydroxy-3, 3a, 8a-trimethyl-3, 3a, 8, 8a-tetrahydro-2H-furo[2,3-b]-indol-2-one as an antineoplastic agent against mammalian neoplastic cancer cells, presenting marked inhibitory activity on the growth of said neoplastic cells, causing cell death induced by alteration of the cytoplasmic membrane of cancer cells.

### Technical Considerations

The search for new antineoplastic agents has become a race to discover alternatives to existing therapies, which, although it is true that they have proven to be effective for some types of cancer, continue causing various adverse effects in the body, and not in all treated cases cause tumor remission, much less cause complete cell death of the treated neoplastic cells.

Most of the available antineoplastic compounds tend to be extremely aggressive for the normal cells of the body, so their administration must be constantly monitored to be able to deal with the adverse effects caused by them. Likewise, conventional antineoplastic agents have proven to be only useful with specific cell lineages, and it is difficult for them to control the growth of very resistant cell lineages such as those tumors generated from central nervous system tissue, which have little effective treatment with available antineoplastic compounds such as cis-platinum, which it should be noted is used in high doses to generate neoplastic cells growth inhibition, capable of producing tumor masses remission.

Many of the current research lines have been aimed at testing compounds commonly used in humans, designed for other pathologies as a source of raw material for obtaining new antineoplastic compounds. However, most of the evaluated compounds turn out to be little potent as cytotoxic agents, so their use is limited to serving as adjuvants to the available chemotherapies.

One of the approaches to searching for new antineoplastic compounds that has made the least progress is to search for new candidates to serve as antineoplastic compounds among biocidal compounds intended for non-therapeutic purposes. This is largely due to the fact that these compounds are rejected because they are designed for the control of parasitic organisms with a phylogenetic origin different from that of mammals and primates. Therefore, it is very unlikely that among these compounds there will be a suitable candidate to serve as an antineoplastic agent in mammals since their action mechanisms are linked to metabolic pathways and structures that are not present in the animal cells of vertebrates.

Although it is true that it is unlikely to generate new antineoplastic agents from biocidal compounds intended for the agricultural sector, there are some new compounds that could be tested, since so far they have not reported to have any type of adverse effect on animal cells. For example, Andranone is a compound that has been described not long ago in the Patent application MX/a/2020/007930. Said compound is an antifungal agent that causes membrane disruption of fungal cells of the main agents phytopathogens, by binding to the sterols present in the cytoplasmic membrane in an irreversible manner. Said compound has a strong inhibitory activity on the growth and development of phytopathogenic fungi such as Fusarium spp, Rhizoctonia spp, Moniliophthora spp, Alternaria spp, Colletotrichum lindemuthianum, Colletotrichum gloeosporioides, Bipolaris spp, Verticillium spp and Mycosphaerella fijiensis, and to date it has not been reported to have any other type of effect on animal cells, and in studies presented in Patent application above mentioned, it was demonstrated that Andranone is harmless against free-living organisms. Thus, from these data, it is possible to deduce that said compound is harmless on animal and/or plant cells. However, due to its action mechanism as fungicide agent so specific, derived from its interaction with sterols of the fungal cell membrane, it is very unlikely that it was taken as a starting point for obtaining a new antineoplastic agent.

In view of the above, it is clear that the anticancer compounds available on the market are not entirely effective as antineoplastic agents against most resistant cancers, such as those of the central nervous system. Likewise, most of the compounds available on the market require high doses to cause an appreciable decrease in cell growth of the treated neoplastic cells.

In view of the above problems, it is necessary to provide alternative compounds for the treatment of cancer in mammals, particularly in humans, which produce an active remission of cancer cells growth. Furthermore, there is a need to provide alternatives for the treatment of various cancers, which are specific, with novel action mechanisms and whose concentrations required to achieve inhibition and/or arrest of cancer cells growth, are lower than those required by the currently used conventional treatments.

### SUMMARY

In order to overcome the limitations of conventional compounds for the treatment of cancer in animal cells, the present disclosure aims to provide a new anticancer agent against neoplastic animal cells, with a specific and novel action mechanism, achieving a minimum inhibitory concentration of the growth and development of cancer cells less compared to those deployed by current conventional treatments and finally, whose adverse effects on the administered organisms be null.

Another object of the present disclosure is to provide an anticancer drug against mammalian neoplastic cells, which is specifically effective in hominids, preferably in humans.

A further object of the present disclosure is to provide an antineoplastic medicament adapted to be administered intravenously or intradermally.

A further objective of the present disclosure is to provide antineoplastic compositions that can be stored for prolonged periods of time at room temperature, without showing degradation of their active ingredient.

The aforementioned objectives, as well as others and the advantages of the present disclosure, will become apparent from the following detailed description thereof.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows the fluorescence analysis results of the effect of Andranone and cis-platinum on the cell membrane of cells of the U-251 cell line;

FIG. 2 shows the graphs obtained from the Annexin-V and Propidium Iodide model assessed by flow cytometry, of the effect of Andranone and cis-platinum on cultures of the U-251 cell line;

FIG. 3 shows a graph showing the average frequency of micronuclei in the culture of human peripheral blood lymphocytes exposed in vitro to different concentrations of Andranone, in which each bar represents the mean±SD of 3 independent experiments (N=15); and

FIG. 4 shows a graph in which the changes in expression of the cytokines (A) TNF-α, (B) TGF-β and (C) the RelA subunit of the transcription factor NF-κB in lymphocyte cultures of human peripheral blood exposed in vitro to different Andranone concentrations.

### DETAILED DESCRIPTION

The present disclosure relates to a new antineoplastic agent against neoplastic animal cells and more specifically, it provides an indole alkaloid that causes cell death induced by alteration of the cell membrane at low doses on cell lines resistant to other conventional anticancer compounds such as cis-platinum.

It is known that the indole alkaloid (3S, 3aR, 8aS)-3-butyl-5-hydroxy-3, 3a, 8a-trimethyl-3, 3a, 8, 8a-tetrahydro-2H-furo [2, 3-b]-indol-2-one represented by formula I, generically known as Andranone, has been shown to have a specific fungicidal effect.

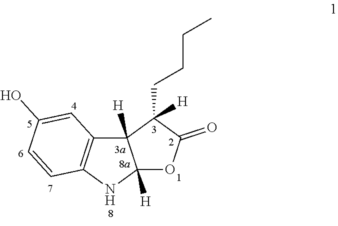 [SMILES: CCCC[C@@H]1C(=O)O[C@@H]2Nc3ccc(O)cc3[C@H]12]

This alkaloid has proven to be effective as an antifungal compound, causing membrane disruption by binding to the sterols present in the cytoplasmic membrane in an irreversible manner. Therefore, said compound has only been shown to be able to bind to a specific component present only in fungal cells, so in no way would it have been expected to present any type of specific interaction with other types of eukaryotic cells.

In the studies carried out in the present disclosure, it has been found that, contrary to all reasonable expectations, animal neoplastic cells, in particular cancer cells of hominids such as humans, have surprisingly turned out to be susceptible to the Andranone action, which causes cell death induced by cell membrane alteration. Andranone, unlike other compounds used as antineoplastic agents, presents high cytotoxicity at low doses on cell lines resistant to other conventional anticancer compounds such as cis-platinum, which has been shown not to be effective in the treatment of resistant cancers such as brain cancer.

The Andranone of the present disclosure can be used as a medicine or in the manufacture of medicines for the treatment of cancer, adapted to be administered intravenously or intradermally, and to provide preferred plasma concentrations between 0.1 and 1.0 μM, more specifically between 0.10 and 0.6 μM and more preferably between 0.1 and 0.3 μM.

Preferably, the Andranone compound of the present disclosure is used in compositions comprising carbopol to increase the solubility of Andranone, so that the active ingredient increases its bioavailability in the body. Said compositions comprise between 10 and 40% (w/v) of Andranone, and between 0.05 and 0.1% (w/v) of carbopol, dissolved in deionized water. The compositions are placed into vials that can be stored at temperature between −4 and 25° C. for a period of up to 12 months. In this way, the Andranone compositions of the present disclosure can provide effective plasma concentrations of Andranone for the control of tumor masses.

Additionally, the compositions of the present disclosure have been shown to increase the bioavailability of Andranone at the plasma level, making them useful as a treatment for aggressive cancers such as brain cancer, since a constant effective concentration of the active ingredient can be maintained easily with small doses.

Likewise, the present disclosure provides a method of cancer treatment, which allows reducing the size of pre-existing tumor masses in mammals, which consists of administering, intravenously or intradermally, a therapeutically effective amount of Andranone or the Andranone composition with carbopol, being such amount therapeutically effective, one amount such that it allows providing a plasma concentration between 0.1 and 1.0 μM of Andranone at plasma level.

To demonstrate the antineoplastic effect of Andranone, tests were carried out to assess the cytotoxicity of the compound on cancer cell lines and to elucidate its action mechanism, as shown in the examples described below.

### Example 1. Antineoplastic Activity of Andranone

For the evaluation of the cytotoxic effect of Andranone on neoplastic cells, 6 cell lines were used:

HCT-15 (colon), MCF-7 (breast), K-562 (leukemia), U-251 (central nervous system), PC-3 (prostate), SKUL (lung) and COST-7 (cervical uterus) from the National Cancer Institute (NCI) of the United States. The cytotoxicity of Andranone was determined in sixfold microcultures using preferable Andranone concentrations between 0.1 and 1.0 μM, more specifically between 0.1 and 0.6 μM and mostly between 0.1 and 0.3 μM. Cell viability and growth were measured indirectly by the sulforhodamine B method according to procedures validated by the NCI, using salicylic acid and Mezalasin as negative control compounds, and Cis-platinum as a control antineoplastic drug at concentrations of 100 μM. The obtained results are described in Table 1.

TABLE 1
Determination of the cytotoxic effect of Andranone
on different neoplastic cell lines.

| | | Growth inhibition by cell line (%) | | | | | | |
|---|---|---|---|---|---|---|---|---|
| COMPOUND | C/μM | HTC-15 | MCF-7 | K-562 | U-251 | PC-3 | SKUL | COST-7 |
| Andranone | 0.1 | 65.5 | 52.6 | 39.8 | 72.2 | 69.8 | 64.8 | 59.4 |
| | 0.2 | 99.2 | 97.8 | 96.4 | 98.2 | 91.6 | 96.4 | 93.9 |
| Salicylic acid | 100 | NC | 4.9 | 6.6 | 6.8 | 6.9 | 10.1 | 11.2 |
| Mesalazine | 100 | NC | 11.9 | 8.1 | 6.3 | 3.1 | NC | NC |
| Cisplatin | 100 | 87.49 | 42.65 | 79.15 | 65.9 | 32.42 | 81.35 | 43.39 |

### NC: Non-Cytotoxic

As can be seen in the results shown in Table 1, Andranone presents a high cytotoxicity in all the cancer cell lines evaluated, observing that at concentration of 0.2 μM, all the treated cell lines presented a greater inhibition in their growth compared to the cis-platinum which was used as a control antineoplastic drug. In all cases, Andranone demonstrated greater cytotoxicity than cis-platinum on all cell lines, even at lower concentrations than those conventionally used for the control antineoplastic compound.

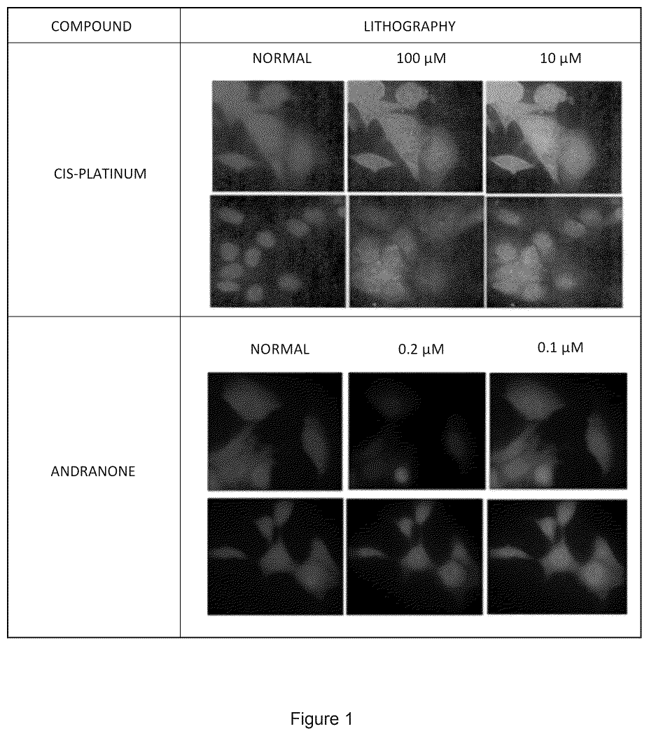

On the other hand, the Andranone cytotoxic effect was corroborated when analyzing the total death of the cells in an Annexin-V and propidium iodide model, assessed by flow cytometry, in which were observed U-251 cells treated with Cis-platinum 10, 50 and 100 μM, and treated with 0.15, 0.16, 0.18, 0.19 and 0.20 μM of Andranone. As can be seen in FIG. 1, a marked shift of the phases from highest to lowest and from quadrant 3 to quadrants 2 and 4 is observed, which indicates that the U-251 cells treated with Andranone are completely dead and not moribund in all assessed concentrations.

### In Vitro Cytotoxic Activity of Andranone

Additionally, the cytotoxic activity was weighted by the IC₅₀ of Andranone on 12 cell lines of neuronal origin, PFSK-1 (brain, cerebral hemisphere); A-172 (Glioblastoma, 53 years old patient); SW1088 (Brain Fibroblast); Daoy (Brain, Cerebellum); 1321N1 (Normal brain cells) HT-29 (Colorectal Carcinoma); SW-620 (Colorectal carcinoma); H-358 (Lung carcinoma); A-549 (Lung carcinoma); LA-N-5 (Neuroblastoma); SK—N-SH (Neuroblastoma); and L-929 (Mouse fibrosarcoma) using Bevacizumab as a control. The IC₅₀ determination of Andranone was carried out in microcultures in sixfold. The results of the IC₅₀ assessment are shown in Table 2.

TABLE 2
Cellular cytotoxicity data, represented by the minimum
inhibitory concentration in different cell lines.

| | |
|---|---|
| Andranone | Bevacizumab |

| Cell line | IC₅₀ (μM) | DEST | IC₅₀ (μM) | DEST |
|---|---|---|---|---|
| PFSK-1 | 2.45 | 0.6 | 31.23 | 0.6 |
| A-172 | 3.19 | 0.59 | 41.4 | 0.59 |
| SW1088 | 3.67 | 2.3 | 47.27 | 2.3 |
| Daoy | 1.85 | 13 | 29.43 | 13 |
| 1321N1 | 46.31 | 0.76 | 39.5 | 0.76 |
| HT-29 | 17.74 | 0.91 | 38.43 | 0.91 |
| SW-620 | 19.56 | 2.5 | 27.18 | 2.5 |
| H-358 | 14.69 | 3 | 115.2 | 3 |
| A-549 | 10.77 | 1.2 | 105.4 | 1.2 |
| LA-N-5 | 11.28 | 0.89 | 79.32 | 0.89 |
| SK-N-SH | 21.51 | 1.55 | 88.39 | 1.55 |
| L-929 | 12.82 | 0.11 | 125.8 | 0.11 |

The results show that Andranone has a smaller IC₅₀ than that obtained with Bevacizumab, so it can be seen that Andranone is much more powerful against the evaluated cell lines.

### Example 2. Determination of Action Mechanism of Andranone

To determine the Andranone action mechanism on neoplastic cells, a fluorescence analysis was performed on cells of the U-251 cell line treated with 10 and 100 μM of cis-platinum and 0.1 and 0.2 μM of Andranone.

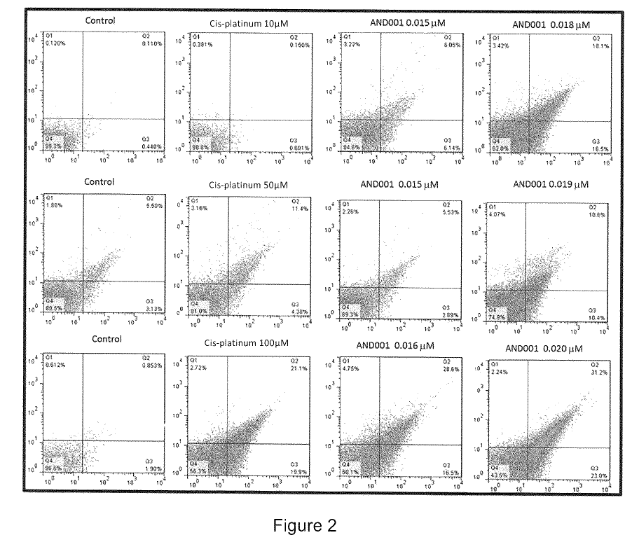

As can be seen in FIG. 2, in the controls without application (Normal) a sustained blue color can be seen, indicating cellular integrity in both treatments. In the high and low concentration of Cis-platinum, an alteration in color is observed, indicating that there was a cell membrane alteration without being so obvious. However, for Andranone in high concentration (0.2 μM) a marked red color is observed, indicating the total death of the cancer cell, which is observed in cells that have undergone a cell death process induced by cell membrane alteration.

From the results obtained in the examples described above, it can be concluded that the Andranone compound presents a marked cytotoxic effect on cancer cell lines, which is much greater than that observed when using Cis-platinum. Furthermore, it is observed that Andranone is capable of inhibiting the cell growth of very resistant neoplastic cell lines such as U-251 brain cancer cell line, which has been shown not to be treated efficiently with conventional anticancer compounds such as Cis-platinum.

### In Vitro Evaluation of Andranone Toxicity in Human Peripheral Lymphocytes.

500 μL of heparinized peripheral blood from a young, healthy donor was incubated with RPMI 1640 medium supplemented with phytohemagglutinin (2%) and penicillin-streptomycin (0.5%) to a final volume of 2.5 mL with different concentrations of Andranone (2×10⁻⁶, 4×10⁻⁶, 8×10⁻⁶, 12×10⁻⁶, and 16×10⁻⁶ mM), as well as the positive controls (Bleomycin, 3.2 μg/mL) and the negative control (lymphocytes without Andranone) under the same conditions. At 44 h, cytochalasin B (4.5 μg/mL) was added to all cultures and at 72 h the lymphocytes were harvested by centrifugation at 1500 rpm for 10 min. The cell bud was divided into 2 parts, one to perform the micronucleus test and the other was kept in PBS (pH 7.5) at −80° C. to perform the RT-qPCR tests.

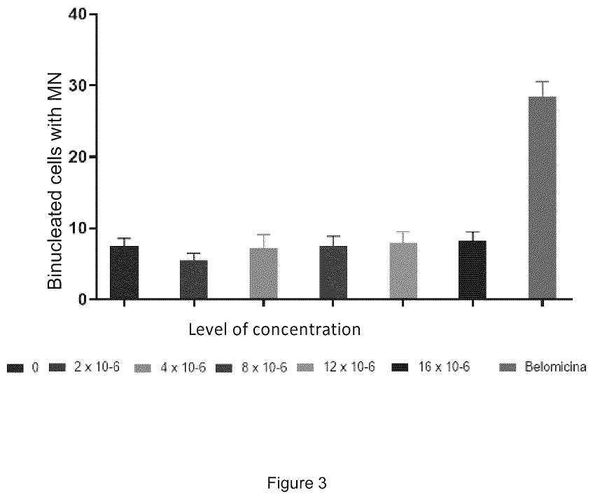

For the micronucleus test, the first part of the cell bud was prefixed with 1 mL of Carnoy's fixative (3:1) and centrifuged at 1500 rpm for 10 min. The supernatant was removed, and the cell bud was resuspended. 5 mL of Carnoy fixative was added and centrifuged again at 1500 rpm for 10 min. This step was repeated 2 more times. Lamellae were made on slides using the drip technique and allowed to air dry. The samples were stained in 5% Giemsa dye and analyzed in the optical microscope at 40× to determine the frequency of micronuclei in 1000 cells (in duplicate). The results are shown in FIG. 3. As seen in the graph, the concentrations of 2×10⁻⁶, 4×10⁻⁶, 8×10⁻⁶, 12×10⁻⁶, and 16×10⁻⁶ mM of Andranone did not significantly increase the frequency of cells linked with micronuclei compared to the negative control (p<0.05). However, Bleomycin used as a positive control significantly increased the frequency of MN.

Table 3 shows the averages of the IDC cell division indices of proliferation with cytokinesis blocks (IPBC) as well as the percentage of relative cytotoxicity, calculated from the micronucleus assay for lymphocyte cultures exposed to different Andranone concentrations.

TABLE 3
Cytotoxic effect in cultures of human peripheral
blood lymphocytes exposed in vitro to Andranone.

| | | | Relative cytotoxicity |
|---|---|---|---|
| Andranone (mM) | IDN | IPBC | (%) |
| 0 | 1.86 ± 0.02 | 1.85 ± 0.03 | — |
| 2 × 10⁻⁶ | 1.78 ± 0.04 | 1.71 ± 0.04 | 4.91 |
| 4 × 10⁻⁶ | 1.82 ± 0.05 | 1.83 ± 0.03 | 1.12 |
| 8 × 10⁻⁶ | 1.83 ± 0.24 | 1.84 ± 0.08 | 0.99 |
| 12 × 10⁻⁶ | 1.79 ± 0.05 | 1.79 ± 0.08 | 3.02 |
| 16 × 10⁻⁶ | 1.80 ± 0.07 | 1.78 ± 0.01 | 3.63 |
| Bleomycin | | | |
| 3.2 mg/mL | 1.60 ± 0.06 | 1.56 ± 0.05 | 34.50 |

Both IDN and IPBC indices of lymphocytes in cultures exposed to 2×10⁻⁶, 4×10⁻⁶, 8×10⁻⁶, 12×10⁻⁶, and 16×10⁻⁶ Mm of Andranone did not show significant differences when compared with the values of the negative control, which demonstrates little cytotoxicity of Andranone on peripheral blood lymphocytes.

To evaluate the expression of TNF-α, TGF-β and the RelA subunit of the NF-κB transcription factor, by RT-qPCR, total RNA was extracted from the second part of human peripheral lymphocytes previously exposed to 5 Andranone concentrations using the Trizol reagent. 100 μL of Trizol was added to each cell bud and allowed to rest for 5 min at room temperature. 60 μL of chloroform was added, it was shaken and allowed to incubate for 3 min at room temperature. The samples were then centrifuged. The aqueous phase was transferred to a tube with 150 μL of isopropanol and 1 μL of glycogen. It was incubated for 24 hours at 4° C. After that time, it was centrifuged at 10,000 rpm for 10 minutes. The supernatant was decanted and washed with 100 μL of ethanol (75%) by centrifuging for 5 min at 7500 rpm. The supernatant was decanted, and the samples were allowed to dry in a dry bath at 40° C. Finally, the bud was resuspended in 10 μL of nuclease-free water and stored at −80° C. until use.

Once the RNA was extracted, the RNA concentration was quantified in 1 μL of each sample using a spectrophotometer using the 260/280 and 260/230 nm quotients of the absorbances to obtain purity. Later, calculations were carried out, having 200 ng of RNA in each 3 ml of sample. cDNA synthesis was performed from 200 ng of total RNA in a final volume of 200 μL following the Thermo Scientific kit protocol in a BIORAD “Thermal cycler MJ mini” thermocycler with the following conditions: 37° C. for 60 minutes, 70° C. for 10 minutes and 20° C. for 1 minute.

Real-time PCR was performed with the kit on a Stratagen MX300SP Agilent Technologies brand equipment, following the protocol of the Maxima SYBR Green QPCR master mix kit from Thermo Scientific, using SYBR Green as the main fluorochrome and ROX as the reference passive dye. The specific primers for each gene were used to prepare the reaction mixtures. For the amplification assay carried out in an Agilent Technologiesse Stratagen MX300SP thermocycler, the following temperature profile was used: 95° C. for 10 minutes, 50 cycles of 95° C. for 15 seconds and 58° C. for 1 minute. In order to finish with a cycle of 95° C. for 1 minute, 58° C. for 30 seconds and 95° C. for 30 seconds. Each sample was performed in triplicate including a non-tempered control (NTC) and the relative expression levels were analyzed with algorithm 2 using R-actin expression as the endogenous gene. The average values of 3 (N=15) independent experiments were compared with each other using analysis of variance (ANOVA) tests of Newman-KEULS multiple comparison for statistical differences between the averages of the experimental groups and the positive and negative controls, and a linear regression analysis to establish the concentration-effect ratio.

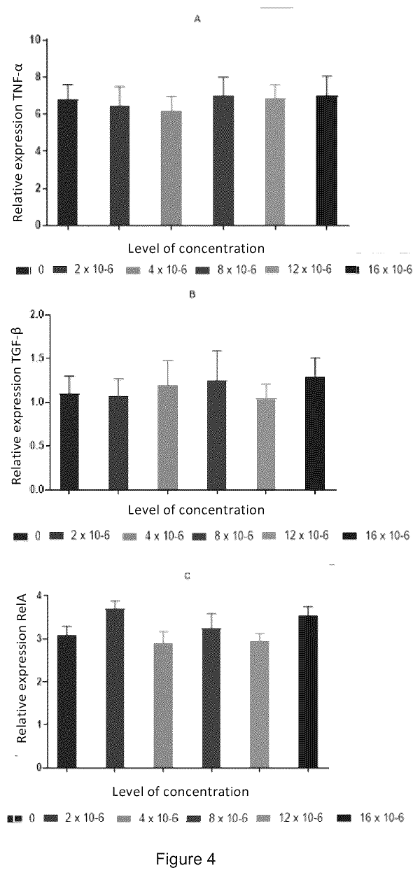

FIG. 4 shows the averages of TNF-α expression in cultures of human peripheral blood lymphocytes exposed in vitro to different concentrations of Andranone. The TNF-α expression, TGF-β and the RelA subunit of the NF-KB transcription factor did not change in human peripheral lymphocytes exposed to any of the Andranone concentrations used in relation to the negative control value. In human lymphocytes exposed in vitro to the compound, there was no increase in any of the transcription factors previously described. Therefore, it is demonstrated that exposure to Andranone does not cause changes in the cytokine expression of peripheral blood lymphocytes.

### Evaluation of In Vivo Antineoplastic Activity

To demonstrate the in vivo antineoplastic effect of Andranone, female Sprague-Dawley rats from the Institute of Cellular Physiology Vivarium of the UNAM were used. Fifty-day-old rats weighing between 150 and 180 g were selected. All animals were housed in a temperature-controlled room at 25±1° C. with a 12-h light/dark cycle in the laboratory animal facility. The animals were fed with food and water ad libitum. Before the experiments, the rats were acclimatized for 7 days to adapt to the laboratory environment. All procedures were approved by the Animal Experimentation Ethics Committee (Ref. 30/55 and Ref. 19/57), Prince of Songkla University, Thailand. The study was divided into two parts, on the one hand, the antineoplastic effect of the Andranone composition with carbopol in already induced tumor masses was assessed, and on the other, the preventive effect of the composition when administered in conjunction with induction of tumor masses was assessed.

To study antineoplastic activity, female Sprague-Dawley rats were divided into six groups, each group consisting of 10 animals. Animals in group I (normal) did not receive treatment. Rats in groups II (negative control) and III to VI were injected with NMU (N-nitroso-N-methylurea from Sigma Chemical Co), to induce breast cancer at 50, 80 and 110 days old. Furthermore, group III (vehicle) received only the vehicle, while groups IV, V and VI were treated with 10, 20 and 40 mg/kg body weight of the present disclosure composition (ANR), respectively. Treatments were started when the first tumors had a diameter ≥5 mm. All rats received their specific treatment every 2 days until 30 days after the first tumor detection.

In order to study cancer preventive activity, female Sprague-Dawley rats were divided into five groups, each group consisting of 30 animals. Animals in group I (normal) did not receive treatment. Animals in Groups II to IV were injected I.V. 50 mg/kg NMU (N-nitroso-N-methylurea from Sigma Chemical Co) to induce breast cancer at 50, 80 and 110 days old. Group II (control) received a distilled water and Tween-80 (vehicle) mixture. Groups III, IV and V were treated with vehicle (group III) and the composition of Andranone (ANR) at 10 and 20 mg/kg body weight (groups IV and V respectively), 14 days after the first application of NMU. Groups II, III and IV were treated three times a week for 76 days. Tumor size, body weight, and clinical signs were observed and registered.

All rats were sacrificed at the end of the experiment. The wet tumor and organs, such as lung, liver, kidney, heart, and gastric tissues, were measured and weighed. The rate between the weight of the organs and the final body weight and their values were expressed as a percentage. For paired organs, the mean weight of the two organs was used to calculate the organ weight/body weight ratio. Blood samples were obtained from the rats by cardiac puncture for hematological and biochemical analysis. The test results of antineoplastic and preventive effect are shown in Table 4.

TABLE 4
Results of the antineoplastic and preventive evaluation of the Andranone
composition with carbopol in a Sprague-Dawley rat model.

| | Body | | Tumor | | | | | |
|---|---|---|---|---|---|---|---|---|
| | weight | | weight (g) | | | | | |
| | (X ± | Tumor | (X ± | | | | | |
| | DEST, | incidence | DEST, | Organ weight/Body weight (%) | | | | |
| Group | n = 30) | (%) | n = 30) | Heart | Liver | Lung | Spleen | Kidney |

ANTI-CANCER

| | | | | | | | | |
|---|---|---|---|---|---|---|---|---|
| Normal | 281.33 ± | — | — | 0.37 ± | 3.62 ± | 0.43 ± | 0.27 ± | 0.60 ± |
| | 12.54 | | | 0.02 | 0.19 | 0.02 | 0.03 | 0.06 |
| Control | 195.50 ± | 100 | 5.82 ± | 0.38 ± | 4.84 ± | 0.55 ± | 0.52 ± | 0.81 ± |
| | 12.73 | | 1.38 | 0.04 | 0.72 | 0.11 | 0.22 | 0.15 |
| Vehicle | 188.40 ± | 100 | 5.02 ± | 0.41 ± | 4.69 ± | 0.52 ± | 0.36 ± | 0.69 ± |
| | 11.43 | | 1.07 | 0.06 | 0.17 | 0.07 | 0.16 | 0.09 |
| ANR 10 | 288.32 ± | 35 ± 6 | 3.86 ± | 0.38 ± | 4.09 ± | 0.50 ± | 0.41 ± | 0.67 ± |
| | 12.46 | | 0.85 | 0.05 | 0.17 | 0.06 | 0.16 | 0.07 |
| ANR 20 | 283.56 ± | 12 ± 4 | 2.06 ± | 0.37 ± | 3.74 ± | 0.53 ± | 0.29 ± | 0.69 ± |
| | 22.75 | | 0.78 | 0.04 | 0.06 | 0.09 | 0.06 | 0.08 |
| ANR 40 | 289.50 ± | 5 ± 1 | 1.14 ± | 0.39 ± | 3.61 ± | 0.53 ± | 0.26 ± | 0.68 ± |
| | 12.44 | | 0.96 | 0.07 | 0.21 | 0.09 | 0.22 | 0.05 |

PREVENTIVE

| | | | | | | | | |
|---|---|---|---|---|---|---|---|---|
| Normal | 281.33 ± | | | 0.37 ± | 3.62 ± | 0.43 ± | 0.27 ± | 0.60 ± |
| | 12.54 | | | 0.02 | 0.19 | 0.02 | 0.03 | 0.06 |
| Control | 205.50 ± | 100 | 5.82 ± | 0.38 ± | 4.84 ± | 0.55 ± | 0.52 ± | 0.71 ± |
| | 12.73 | | 1.38 | 0.04 | 0.72 | 0.11 | 0.22 | 0.15 |
| Vehicle | 219.80 ± | 100 | 3.05 ± | 0.36 ± | 4.35 ± | 0.50 ± | 0.51 ± | 0.75 ± |
| | 12.46 | | 1.52 | 0.03 | 0.47 | 0.07 | 0.09 | 0.04 |
| ANR 10 | 290.50 ± | 20 ± 4 | 0.95 ± | 0.38 ± | 3.38 ± | 0.42 ± | 0.27 ± | 0.64 ± |
| | 15.30 | | 0.15 | 0.02 | 0.29 | 0.03 | 0.03 | 0.04 |
| ANR 20 | 294.00 ± | 10 ± 0.8 | 0.88 ± | 0.39 ± | 3.48 ± | 0.49 ± | 0.32 ± | 0.64 ± |
| | 14.80 | | 0.28 | 0.06 | 0.54 | 0.05 | 0.10 | 0.06 |

The results showed a significant reduction in the weight and size of the tumor masses in the treated groups. Likewise, in prevention tests a much smaller size of the induced tumor masses is observed than in the control groups, as well as a lower incidence of neoplastic growths. The above demonstrated that, with the administration of Andranone, it is possible to cause the regression of tumor masses in vivo and also prevent the new tumor masses appearance.

The present disclosure has been described according to a preferred, but non-limiting, embodiments; however, it will be apparent to a person skilled in the art that modifications may be made to the disclosure, without departing from its spirit and scope.

1. A compound of general formula I, 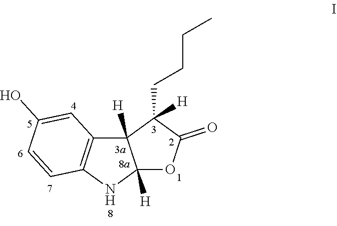
as an antineoplastic agent.

2. The compound of general formula I, wherein the compound has antineoplastic activity.

3. The compound according to claim 1, as a medicine.

4. The compound according to claim 1, for the treatment of cancer in mammals.

5. The antineoplastic compound according to claim 4, wherein the mammal is a hominid.

6. The compound according to claim 5, wherein the hominid mammal is a human.

7. The compound according to claim 4, wherein the cancer is selected from the group consisting of colon cancer, breast cancer, leukemia, central nervous system cancer, prostate cancer, lung cancer and cervical cancer.

8. The compound according to claim 7, wherein the cancer is cancer of the central nervous system.

9. The compound according to claim 1, wherein the compound is adapted to be administered intravenously or intradermally.

10. The compound according to claim 1, wherein the compound is adapted to provide compound concentrations of 1.0 to 1.0 μM.

11. The compound according to claim 1, wherein the compound alters the cytoplasmic membrane of cancer cells, inducing their cell death.

12. An antineoplastic composition comprising 10 to 40% (w/v) of the compound of claim 1; 0.05 to 0.1% (w/v) carbopol; and deionized water.

13. The antineoplastic composition according to claim 12, wherein the composition is adapted to provide plasma compound concentrations of general formula (I) of 0.1 and to 6 μM.

14. The composition according to claim 12, as a medicine for the treatment of cancer.

15. The composition according to claim 14, wherein the medicine is a medicine for intravenous or intradermal application.

16. The composition according to claim 14, wherein the cancer is selected from the group consisting of colon cancer, breast cancer, leukemia, central nervous system cancer, prostate cancer, lung cancer and cervical cancer.

17. A method for treating cancer in a subject, comprising administering intravenously or intradermally a therapeutically effective amount of the compound of claim 1.

18. The treatment method according to claim 17, wherein the therapeutically effective amount is an amount sufficient to provide a plasma concentration of 0.1 to 1.0 μM of the compound of general formula I.

19. A method for treating cancer in a subject, comprising administering intravenously or intradermally a therapeutically effective amount of the composition of claim 12.

20. The compound according to claim 1, wherein the compound is adapted to provide compound concentrations of 0.1 to 0.6 μM.
